(19) United States
(12) Patent Application Publication
(10) Pub. No.: US 2015/0182121 A1
(43) Pub. Date: Jul. 2, 2015

(54) LOW-COST SCREENING SYSTEM FOR BREAST CANCER DETECTION

(71) Applicant: THE RESEARCH FOUNDATION FOR THE STATE UNIVERSITY OF NEW YORK, Albany, NY (US)

(72) Inventors: Randall L. Barbour, Glen Head, NY (US); Rabah M. Al Abdi, Brooklyn, NY (US); Harry Graber, Brooklyn, NY (US)

(73) Assignee: THE RESEARCH FOUNDATION FOR THE STATE UNIVERSITY OF NEW YORK, Albany, NY (US)

(21) Appl. No.: 14/415,839

(22) PCT Filed: Jul. 19, 2013
(86) PCT No.: PCT/US13/51276

§ 371 Date: Jan. 20, 2015

Related U.S. Application Data
(60) Provisional application No. 61/674,029, filed on Jul. 20, 2012.

(51) Int. Cl. A61B 5/00

(52) U.S. Cl. CPC A61B 5/0091; A61B 5/0073; A61B 5/6804; A61B 5/742; A61B 5/0013; A61B 5/7282

(57) ABSTRACT

A portable and wearable tumor detector includes a brassier and devices for enabling optical tomography in a non-clinical setting. Light emitting devices and light sensing devices are provided on the brassier, and a controller for performing a tomographic scanning is attached to the brassier. A computing means and a communication means may be provided to generate at least one tomographic image. Each of the two breasts under examination can be employed as a reference structure for generating a tomographic image for the other of the two breasts, thereby providing a self-referencing image generation mechanism. The images and/or data can be reviewed by the subject of the tomographic scanning or by a medical professional. The tomographic scanning can be performed at any location if provided with a portable power supply system.

### CROSS-REFERENCE TO RELATED APPLICATIONS

This application claims the benefit of U.S. Provisional Application No. 61/674,029, filed on Jul. 20, 2012.

### STATEMENT REGARDING FEDERALLY SPONSORED RESEARCH OR DEVELOPMENT

This invention was made with government support under Contract No. R41CA096102 awarded by the National Institute of Health, Contact No. DAMD017-03-C-0018 awarded by U.S. Army, Contract No. NYSTAR-TIPP C020041 awarded by New York State Foundation for Science, Technology, and Innovation—Technology Transfer Incentive Program, Contract No. IMG0403022 to Susan G. Komen Foundation, and a grant to Brooklyn Hospital Center from New York State Department of Health through Empirical Clinical Research Investigator Program. The federal government of the United States has certain rights in the invention.

### FIELD OF THE INVENTION

The present invention relates to a wearable apparatus for breast cancer detection and a method for operating the same.

### BACKGROUND OF THE INVENTION

Key to the utility of disease screening methods is the need for high sensitivity to the presence of the disease while employing economical sensing strategies. In the case of detection of breast cancer, the leading screening method is x-ray mammography. See Fletcher, S. W., & Elmore, J. G. (2003) Clinical practice: Mammographic screening for breast cancer, The New England journal of medicine 348: 1672-1680. While effective for the detection of most cancers of the breast, the sensitivity of this technique is limited by small sized tumors, especially in women with dense breasts. See Baines, C. J., & Dayan, R (1999) A tangled web: factors likely to affect the efficacy of screening mammography, Journal of the National Cancer Institute 91: 833-838; and Carney, P. A., Miglioretti, D. L., Yankaskas, B. C., Kerlikowske, K., Rosenberg, R., Rutter, C. M., Geller, B. M., Abraham, L. A., Taplin, S. H., Dignan, M., Cutter, G., & Ballard-Barbash R. (2003) Individual and combined effects of age, breast density, and hormone replacement therapy use on the accuracy of screening mammography, Annals of internal medicine 138: 168-175. Also, its use of ionizing sources is a known risk factor for subsequent cancer development, especially in women under age 50. See Ma, H., Hill, C. K., Bernstein, L., & Ursin, G. (2008) Low-dose medical radiation exposure and breast cancer risk in women under age 50 years overall and by estrogen and progesterone receptor status: results from a case-control and a case-case comparison, Breast Cancer Res Treat 109: 77-90; and Nekolla, E. A., Griebel, J., & Brix, G. (2008) Radiation risk associated with mammography screening examinations for women younger than 50 years of age, Z Med Phys 18: 170-179. Still another consideration impacting its use is the cost of the instrumentation. Today digital x-ray mammography systems typically cost several hundred thousand dollars and require considerable skill in the interpretation of the resultant images. Other imaging methods are available, but these either lack the sensitivity required of screening methods, are operator dependent (e.g., ultrasound imaging) or employ costly instrumentation (e.g., magnetic resonance imaging (MRI)). Nevertheless, the fact that these methods can yield high resolution images favors their continued use.

An alternative approach to structural imaging methods is methods that exploit expected functional differences caused by the presence of the disease. A common example is F-19 labeled glucose used in PET imaging for the detection of tumor metastasis. See Avril, N., Bense, S., Ziegler, S. I., Dose, J., Weber, W., Laubenbacher, C., Romer, W., Janicke, F., & Schwaiger, M. (1997) Breast imaging with fluorine-18-FDG PET: quantitative image analysis. Journal of nuclear medicine: official publication, Society of Nuclear Medicine 38: 1186-1191; and Rosen, E. L., Eubank, W. B., & Mankoff, D. A. (2007) FDG PET, PET/CT, and breast cancer imaging. Radiographics: a review publication of the Radiological Society of North America, Inc 27 Suppl 1: S215-229. This agent exploits the known tendency of tumors to metabolize glucose in an exaggerated way. See Warburg, O., Wind, F., & Negelein, E. (1927) The Metabolism of Tumors in the Body, The Journal of general physiology 8: 519-530.

Another potentially promising functional imaging method is that based on use of near infrared spectroscopic (NIRS) sensing techniques, mainly in the form of diffuse optical measures. This method principally exploits known differences in tumor angiogenesis. Representing a hallmark of tumor growth, the newly formed vessels are present in higher density, and are frequently malformed with dead ends. See Vaupel, P., Harrison, L. (2004) Tumor hypoxia: causative factors, compensatory mechanisms, and cellular response, The oncologist 9 Suppl. 5: 4-9. Other characteristics of tumor vessels include leakiness and sluggish perfusion leading to elevated levels of deoxyhemoglobin and total hemoglobin. See Vaupel & Harrison (2004), supra. Recognition of these features has led to efforts to detect breast tumors based on features of the hemoglobin signal. For example, breast tumor can be detected based on elevated total hemoglobin levels. See Choe, R., Konecky, S. D., Corlu, A., Lee, K., Durduran, T., Busch, D. R., Pathak, S., Czerniecki, B. J., Tchou, J., Fraker, D. L., Demichele, A., Chance, B., Arridge, S. R., Schweiger, M., Culver, J. P., Schnall, M. D., Putt, M. E., Rosen, M. A., & Yodh, A. G. (2009) Differentiation of benign and malignant breast tumors by in-vivo three-dimensional parallel-plate diffuse optical tomography, Journal of biomedical optics 14: 024020; and Wang, J., Jiang, S., Li, Z., diFlorio-Alexander, R. M., Barth, R. J., Kaufman, P. A., Pogue, B. W., & Paulsen, K. D. (2010) In vivo quantitative imaging of normal and cancerous breast tissue using broadband diffuse optical tomography, Medical physics 37: 3715-3724. Further, breast tumor can be detected based on reduced hemoglobin oxygenation. See Choe et al. (2009), supra. Also, breast tumor can be detected based on reduced hemoglobin oxygenation together with other endogenous signatures (e.g., water, lipid content, scattering amplitude, scattering power). See Wang et al. (2010), supra. A noted characteristic of the approach taken in these studies has been the goal to measure the static background optical contrast against which is the enhanced contrast owing to the tumor itself. Whereas a number of reports have indicated promising potential (See Choe et al. (2009), supra.), an elementary challenge of the diffuse optical method in general, is its low spatial resolution. As a consequence, the performance of the method for small tumor detection is notably reduced compared to the structural imaging methods. See van de Ven, S. M., Elias, S. G., Wiethoff, A. J., van der Voort, M., Nielsen, T., Brendel, B., Bontus, C., Uhlemann, F., Nachabe, R., Harbers, R., van Beek, M., Bakker, L., van der Mark, M. B., Luijten, P., Mali, & W. P. (2009) Diffuse optical tomography of the breast: preliminary findings of a new prototype and comparison with magnetic resonance imaging, Eur. Radiol. 19: 1108-1113.

As introduced by Barbour (See Barbour, R. L., Graber, H. L., Pei, Y., Zhong, S., & Schmitz, C. H. (2001) Optical tomographic imaging of dynamic features of dense-scattering media, Journal of the Optical Society of America 18: 3018-3036.), and contrary to the static NIRS imaging methods employed others (See Choe et al. (2009), supra; and Wang, J., Pogue, B. W., Jiang, S., & Paulsen, K. D. (2010) Near-infrared tomography of breast cancer hemoglobin, water, lipid, and scattering using combined frequency domain and CW measurement, Optics letters 35: 82-84), this technology can be adopted to explore dynamic features of tissue optical contrast. This approach has been demonstrated capable of detecting various features related to tumor angiogenesis. As thus far explored, this technique employs a high density sensing array from which is generated a 3D image time series. See Al abdi, R., Graber, H. L., Xu, Y., Barbour R L (2011) Optomechanical imaging system for breast cancer detection, J Optical Society of America A 28: 2473-2493; Flexman, M. L., Khalil, M. A., Al Abdi, R., Kim, H. K., Fong, C. J., Desperito, E., Hershman, D. L., Barbour, R. L., & Hielscher, A. H. (2011) Digital optical tomography system for dynamic breast imaging, Journal of biomedical optics 16: 076014; and Schmitz, C. H., Klemer, D. P., Hardin, R., Katz, M. S., Pei, Y., Graber, H. L., Levin, M. B., Levina, R. D., Franco, N. A., Solomon, W. B., & Barbour, R. L. (2005) Design and implementation of dynamic near-infrared optical tomographic imaging instrumentation for simultaneous dual-breast measurements, Applied optics 44: 2140-2153. In one example, the technique has demonstrated a delayed recovery from an induced respiratory maneuver (Valsalva response) in the tumor bearing breast. See Schmitz, C. H., Löcker, M., Lasker, J. M., Hielscher, A. H., Barbour, R. L. (2002) Instrumentation for fast functional optical tomography, Review of Scientific Instruments 73: 429-439. While potentially promising, the need for high density sensing renders the required instrumentation bulky and costly.

Whereas imaging methods in their various forms are often successful in tumor detection, in all cases their reliance on detection of a focal contrast feature strongly limits their sensitivity to small tumors. An ideal approach to improve sensing capabilities to such targets would be to leverage known disturbances in the molecular machinery of the tumor that will also likely impact intrinsic physical properties of the tissue that are amenable to noninvasive sensing.

### SUMMARY OF THE INVENTION

A portable and wearable tumor detector includes a brassier and devices for enabling optical tomography in a non-clinical setting. Light emitting devices and light sensing devices are provided on the brassier, and a controller for performing a tomographic scanning is attached to the brassier. A computing means and a communication means may be provided to generate at least one tomographic image. Each of the two breasts under examination can be employed as a reference structure for generating a tomographic image for the other of the two breasts, thereby providing a self-referencing image generation mechanism. The images and/or data can be reviewed by the subject of the tomographic scanning or by a medical professional. The tomographic scanning can be performed at any location if provided with a portable power supply system.

According to an aspect of the present disclosure, a tumor detector is provided, which includes: a brassiere conforming to a pair of human breasts; a light emitting device configured to emit light at a plurality of emission points on the brassiere; a light sensing device configured to detect light at a plurality of detection points on the brassiere; and a controller configured to control timing of light emission at the plurality of emission points and to collect data measured by the light sensing device.

In one embodiment, the tumor detector further includes a computing means attached to the brassiere and configured to control a tomographic scan process upon the pair of human breasts. The computing means can be physically attached to the brassiere. Additionally or alternately, the computing means can be configured to generate at least one tomographic image, wherein the at least one tomographic image includes at least one of a two-dimensional map and a three-dimensional map of human breasts.

In another embodiment, the computing means can be configured to generate at least one image for optical density in a breast among the pair of human breasts employing data from another breast among the pair of human breasts as a base line for determining presence or absence of anomalous deviations in optical density in the breast. For example, the computing means can be configured to compare a left-breast image from a left breast within the pair of human breasts with a right-breast image from a right breast within the pair of human breasts, and to generate a map of regions in which local variations in optical density of materials in each of the pair of human breasts are illustrated. For example, the left-breast image can be generated employing data from the right breast as reference data, and the right-breast image can be generated employing data from the left breast as reference data.

In even another embodiment, the computing means can be configured to generate a pair of images corresponding to each of the pair of breasts; and to determine if one of the pair of images differ from another of the pair of images by more than a predefined threshold for identifying presence of a tumor.

According to another aspect of the present disclosure, a method of performing a tomographic scan process is provided. The method includes a step of providing a tumor detector as described above. The method further includes another step of performing a tomographic scan process employing the tumor detector.

According to another aspect of the present disclosure, a method of detecting presence of a tumor in human breasts is provided. The method includes: providing an assembly of a brassier, a light emitting device located on the brassiere, and a light sensing device located on the brassiere, wherein the light emitting device is configured to emit light at a plurality of emission points on the brassiere, and the light sensing device is configured to detect light at a plurality of detection points on the brassiere; disposing the assembly on a pair of human breasts; illuminating the pair of human breasts with infrared radiation from the light emitting device; detecting radiation at the plurality of detection points employing the light sensing device; and comparing a pair of images from the pair of human breasts to determine if one of the pair of images differ from another of the pair of images by more than a predefined threshold for identifying presence of a tumor.

In one embodiment, the method can further include a step of controlling timing of light emission at the plurality of emission points employing a controller, and a step of collecting data measured by the light sensing device employing the controller.

### BRIEF DESCRIPTION OF THE DRAWINGS

FIG. 1 shows reconstructed coronal sections (midway between nipple and chest wall) of unaffected (left) and tumor bearing (right) breast. Dotted line indicates tumor size and position. Image contrast represents Mahalanobis distance computed.

FIG. 2 is a histogram showing group level findings that women with tumor bearing breasts have enhanced vascular pulsatility compared to control groups (Benign, Healthy). Ordinate axis identifies number of pixels having significantly elevated Mahalanobis distance value for identified metrics.

FIG. 3 is a histogram showing group means and standard deviation of the percentage of channels in the tumor-bearing breast that have higher temporal variance of HbOxy than the contralateral healthy breast.

FIG. 4 is a set of schematics for breast illumination according to an embodiment of the present disclosure.

FIG. 5 is a schematic of tumor detector according to an embodiment of the present disclosure.

FIG. 6 is a photo of a LED sources, photodetectors, and a controller that can be employed to form a wearable and portable tumor detector of the present disclosure.

### DETAILED DESCRIPTION OF THE INVENTION

As stated above, the present invention relates to a wearable apparatus for breast cancer detection and a method for operating the same. It is noted that proportions of various elements in the accompanying figures are not drawn to scale to enable clear illustration of elements having smaller dimensions relative to other elements having larger dimensions.

Underlying Theory of the Invention

In the following, an argument is presented that a promising candidate is enhanced vascular pulsatility mediated by nitric oxide in response to known enhanced nitric oxide synthase activity in malignant tumors. See Balestrieri, M. L., Dicitore, A., Benevento, R., Di Maio, M., Santoriello, A., Canonico, S., Giordano, A., & Stiuso, P. (2012) Interplay between membrane lipid peroxidation, transglutaminase activity, and cyclooxygenase 2 expression in the tissue adjoining to breast cancer, Journal of cellular physiology 227: 1577-1582; and Thomsen, L. L., Miles, D. W., Happerfield, L., Bobrow, L. G., Knowles, R. G., & Moncada, S. (1995) Nitric oxide synthase activity in human breast cancer, British journal of cancer 72: 41-44.

One approach previously reported along this line of consideration has been the use of dynamic infrared imaging methods for tumor detection. See Button, T. M., Li, H., Fisher, P., Rosenblatt, R., Dulaimy, K., Li, S., O'Hea, B., Salvitti, M., Geronimo, V., Geronimo. C, Jambawalikar, S., Carvelli, P., & Weiss, R. (2004) Dynamic infrared imaging for the detection of malignancy, Physics in medicine and biology 49: 3105-3116; and Joro, R., Laaperi, A. L., Dastidar, P., Jarvenpaa, R., Kuukasjarvi, T., Toivonen, T., Saaristo, R., & Soimakallio, S. (2009) A dynamic infrared imaging-based diagnostic process for breast cancer, Acta Radiol 50: 860-869. A notable finding from this work is evidence of enhanced vascular pulsations having both a vasomotor and cardiogenic origin in the tumor bearing breast. Also identified by this same group was evidence that inhibition of nitric oxide synthase (NOS) using L-NAME, (N(G)-nitro-L-arginine methyl ester), substantially eliminated the observed enhanced vascular reactivity in tumor bearing rodents indicating that enhanced production of nitric oxide (NO) was the responsible causative agent. See Button et al. (2004), supra. Separately, others have shown that the expression of NOS is significantly elevated in many breast tumors and notable not elevated in many forms of benign breast disease. See Balestrieri et al. (2012), supra; and Thomsen et al. (1995), supra. Also known is that NO is soluble in lipid and has a significantly enhanced lifetime in extracellular fluids. See Thomas, D. D., Liu, X., Kantrow, S. P., & Lancaster, J. R. Jr., (2001) The biological lifetime of nitric oxide: implications for the perivascular dynamics of NO and O₂, Proceedings of the National Academy of Sciences of the United States of America 98: 355-360. Tumor vessels are known for their leakiness. See Ntziachristos, V., Yodh, A. G., Schnall, M., and Chance, B. (2000), Concurrent MRI and diffuse optical tomography of breast after indocyanine green enhancement, Proceedings of the National Academy of Sciences of the United States of America 97: 2767-2772.

It follows that tumor environments may favor a more extensive diffusion of NO well beyond the tumor boarder thus affecting a proportionally larger volume of tissue. This consideration has importance for the detection of small tumors. Separate from appreciation of this potential biomarker for detection of small tumors is an understanding of expected differences in sensitivity to changes in physical properties of tissue resulting from enhanced vascular pulsatility using different sensing methods. As originally noted by, NO induced enhanced pulsations will affect thermo gradients in tissue favoring tumor detection using the method of dynamic infrared imaging. See Anbar, M. (1994) Hyperthermia of the cancerous breast: analysis of mechanism, Cancer letters 84: 23-29; and Button et al. (2004), supra. However, detection of such pulsations can also be accomplished using NIRS methods.

Operational Principles of the Invention:

To achieve an intuitive understanding of the different sensitivities of the NIRS and thermography methods, it is helpful to consider an idealized example: a homogeneous slab medium, with the NIRS measurement comprising illumination of one planar surface and detection of the diffusely transmitted light flux at the other surface, while the thermographic measurement consists of a thermal detector sensing the heat shed across one of the slab surfaces.

In the thermographic imaging case, the flow of heat across the surface is fully determined by the temperature difference between the external medium and the slab's surface layer (this is just Newton's law of cooling). If the temperature of the slab is uniformly increased, then the heat outflow will increase in direct proportion. But it would increase by the same amount if the temperature of only the most superficial part of the slab were somehow increased (this is a simplified model of the thermal effect of cutaneous vasodilation).

A qualitatively different result is found for the sensitivity of the NIR signal to changes in the absorption coefficient. The analytic expression for the exiting flux of diffuse light is F=S exp(−X\sqrt{μₐ/D})/2, where F is the light flux, S is the power per unit areas of the illuminating light, X is the thickness of the slab, and μₐ and D are the slab's homogeneous absorption and diffusion coefficient, respectively. Now if the absorption coefficient is homogeneously modified to a value of μₐ+Δμₐ (where Δμₐ represents vessel pulsatility), the diffuse light flux becomes F+ΔF=S exp(−X{square root over (μₐ+Δμₐ)/D)})/2. If the relative change in μₐ is small (|Δμₐ|≦0.2μₐ, as a rule of thumb), as is typically the case in tissue, then the following expression is an excellent approximation to the relative change in the diffuse light flux:

$$\frac{Δ F}{F} = exp [ - ( \frac{X}{2} \sqrt{\frac{1}{{μ}_{a} D}} ) {Δμ}_{a} ] - 1.$$

That is, the NIRS signal varies exponentially with Δμₐ, thus achieving greater sensitivity than would be expected in the linear-dependence thermography case. A second qualitative distinction between the NIRS and thermography responses also should be noted: since all of the light that is detected has been diffusely transmitted across the entire thickness of the slab, the effect of a homogeneous change in μa on the signal strength is markedly different (greater) from the effect of a change that takes place in only the surface layer.

To conclude, these analytical considerations indicate that compared to thermography measures, NIRS measures (especially transmission measures) will have increased intrinsic sensitivity to NO-induced enhanced vascular pulsations thus favoring the enhanced detection of small tumors.

Returning to the original understanding of conditions needed to achieve an effective screening method (i.e., high sensitivity and low cost), whereas the knowledge of the inherent superiority of NIRS sensing to thermography for detection of pulsations favors the former, experience with these methods has shown that efforts to detect small tumors even with high density sensing hardware has not proven overly effective at least in terms of detecting contrast features strongly localized to the tumor (e.g., enhanced hemoglobin content). However, the understanding that the effected volume of the breast might be significantly extended by effects of NO, argues that detection of small tumors might still prove feasible when measures of vascular pulsatility is made using NIRS, even with low-density sensing arrangements.

Experimental Setup:

To evaluate the possibility discussed above, a series of high density time-series NIRS imaging studies has been conducted on healthy women, women with benign breast disease and women with biopsy-confirmed breast cancer. Two aims of this study were to first document the possibility that the extent of vascular pulsations will extend beyond the known tumor boarder and second that high sensitivity to such behavior can still be retained in highly down-sampled data representing measures achieved using a low density sensing approach.

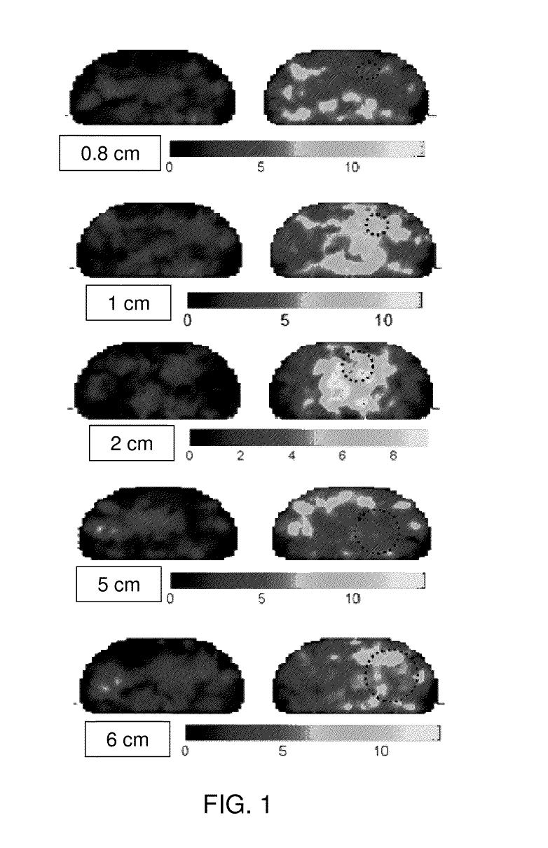

Simultaneous bilateral time series NIRS 3D imaging measurements of the breast were achieved using a conforming articulating sensing array described by Al abdi et al. (2011), supra. Measurements were made with subjects at rest for a period lasting approximately 5 minutes. Shown in FIG. 1 are results obtained from five selected subjects whose tumor size varies from 0.8 to 6 cm in diameter. Displayed contrast feature represents a measure of pulsatility (computed value of the Mahalanobis distance for the amplitude of the temporal standard deviation for deoxyhemoglobin (HbDeoxy) and hemoglobin oxygen saturation (HbSat)) obtained from the volumetric set of image pixel values for the unaffected compared to affected breast. Inspection reveals two notable features. First, the range of enhanced image contrast extends well beyond the known border of the tumor (dotted circle), especially for small tumors. Second, the amplitude of image contrast does not correlate well with tumor size.

Experimental Data:

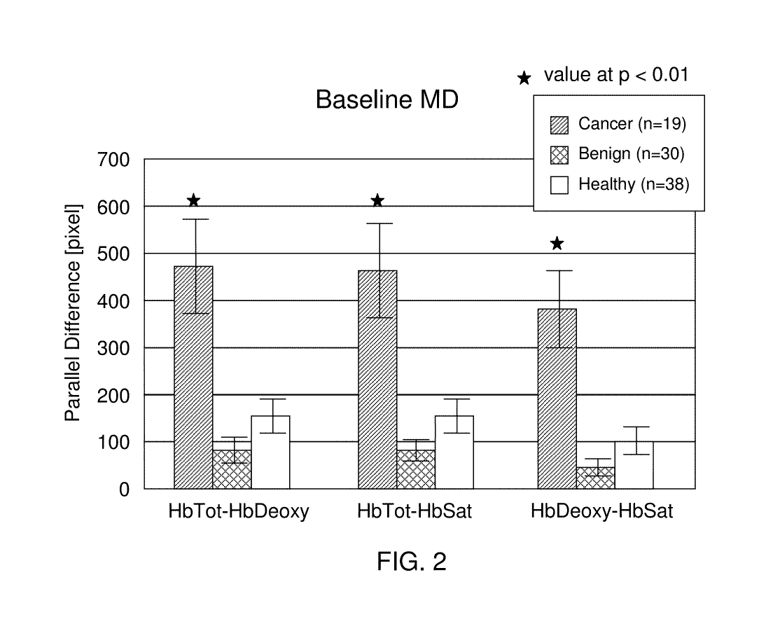

Experimental evidence that the observed finding of a noted bias of enhance pulsations in the tumor bearing breast (shown in FIG. 1) is statistically significant compared to control groups is shown in FIG. 2. Here we have summarized findings from three groups of women; healthy (n=28 subjects), benign (n=30), and tumor (n=19). Inspection shows that the number of image pixels experiencing enhanced pulsatility is significantly increased (p<0.01) in only the affected breast of the tumor bearing subjects. The notation “p<0.01” refers to the value at which the probability becomes less than 0.01.

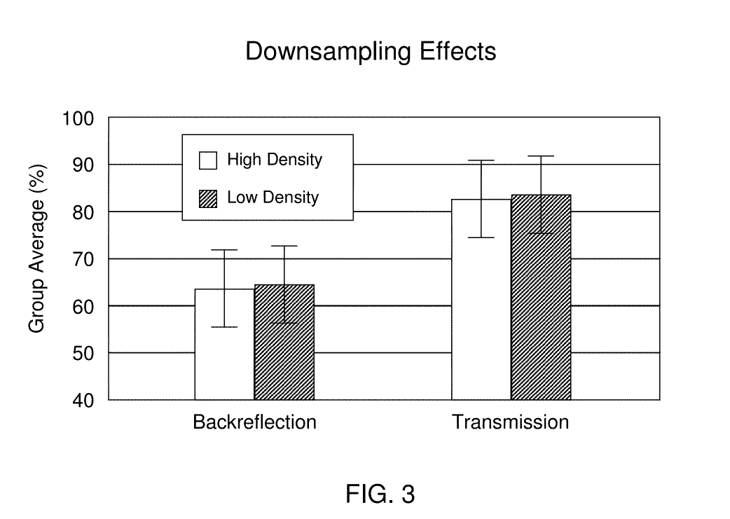

Referring to FIG. 3, results of downsampling on the sensitivity to detecting enhanced vascular pulsatility in the tumor bearing breast are shown. The original data set (high density) comprised nearly 2000 independent optical measurements per image frame per breast evenly distributed between a transmission and backreflection measurement. Shown is fraction (%) of measures for each subset that has an elevated response compared to the unaffected contralateral breast for the five subjects listed in FIG. 1. Two observations are evident. First, as predicted from the above described analytical considerations, the sensitivity of transmission measures are improved over the shorter path length, backreflection measures. Second, the sensitivity to enhanced pulsations is unaffected by downsampling (1% of original data). Certainly, this response is consistent with the observation that the volume of tissue experiencing enhanced pulsatility comprises a significant fraction of the total breast volume, even with small tumors (<1 cm). Appreciation of these findings leads to the sensing approach described subsequently that supports its use as a cost-effective, high sensitivity sensing approach for breast cancer screening.

### Preferred Embodiment

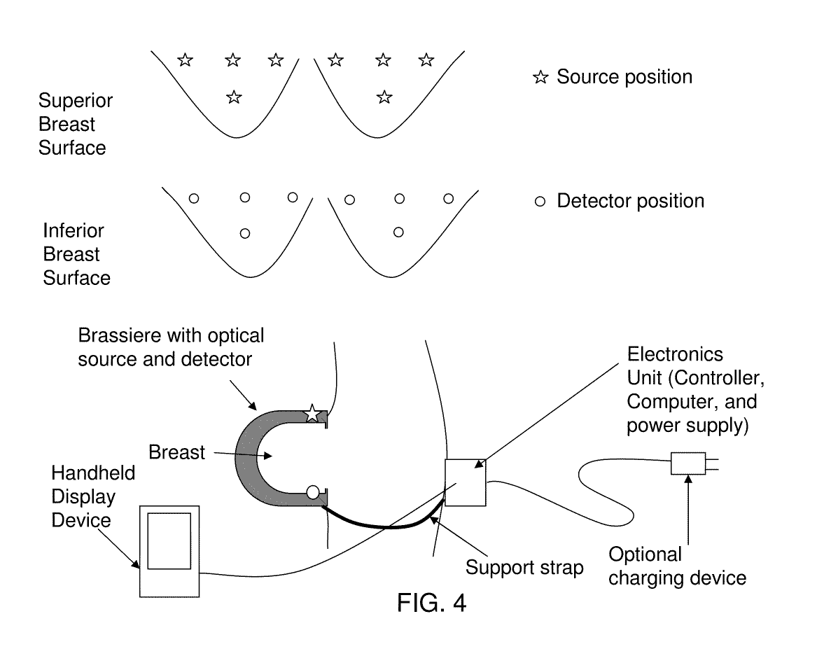

Apart from the clinical diagnostic sensitivity required to support a screening method, its utility in a home or similarly less supervised environment requires access to simplified, low-cost equipment. Illustrated in FIG. 4 is an exemplary tumor detector according to an embodiment of the present disclosure. A limited sensing array is bilaterally incorporated into a dark colored brassiere that conforms to the appropriate chest and breast size for the individual undergoing the optical examination. As used herein, a “brassiere” refers to any structure that a normal female human being can wear around her breast in a manner that covers both of her breasts without a substantial risk of disengagement of the structure from her breasts unless consciously disengaged. The brassier may be portable, which means that the brassier may be taken to any location without regard to mechanical connection or support that is available only at specific locations. The brassier can be employed for self-diagnosis of breast cancer by tomographic imaging of the breasts of a user.

Exemplary positions for emission points (e.g., illumination sources) and detection points (e.g., detectors) are illustrated as stars and circles in FIG. 4, respectively. At least one optical NIR source (4 sources positions illustrated for each cup) is located along the superior aspect of the breast and at least one optical detector (4 detector positions indicated) is located along the inferior surface. Still additional source and detector positions can be considered, but the added information revealed is proportionally less relative to that discerned by the indicated sensing arrangement. Optical measures are accomplished by using at least one emitting wavelength source in the NIR region such as achieved using a LED, or laser diode. Whereas several different light sensing approaches are available to detect reemitted light (continuous wave (CW), frequency domain, time resolved), CW measures involve the least costly and compact sensing arrangement.

An electronics unit is attached to the brassier. The electronics unit can include a controller that controls control timing of light emission at the plurality of emission points and to collect data measured by the light sensing device, a computing means configured to generate at least one tomographic image, and a power supply unit. The controller controls a tomographic scan process upon the pair of human breasts. The power supply unit may be a rechargeable battery, a non-rechargeable battery, or an adapter that converts AC power from an electrical outlet into a DC supply voltage for operation of electronic circuits in the electronics unit. Optionally, a charging device may be provided to enable charging of the power supply unit if the power supply unit includes a rechargeable battery or an adapter. Optionally, the at least one tomographic image generated by the computing means can be displayed on a handheld display device.

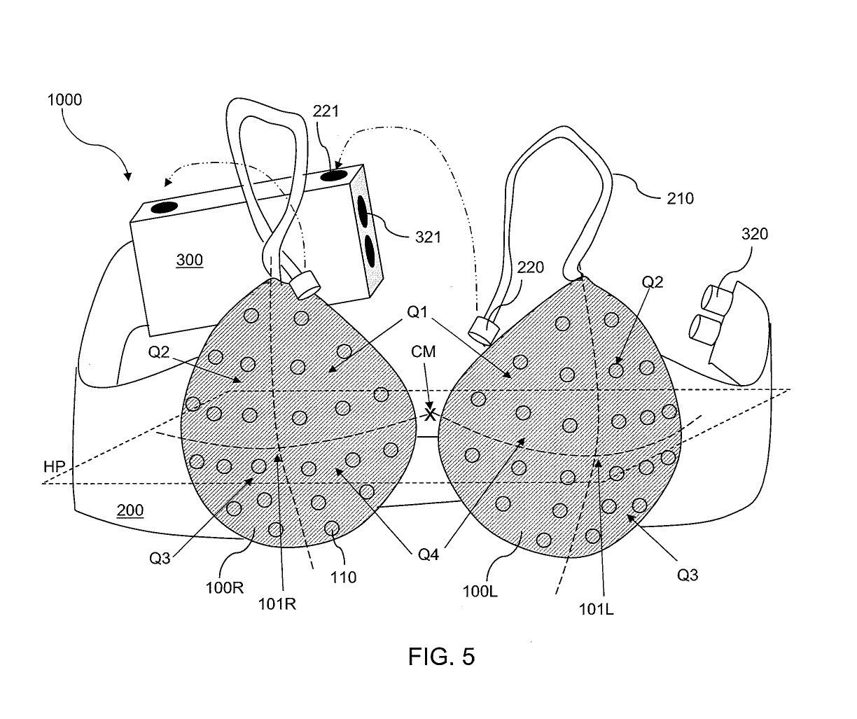

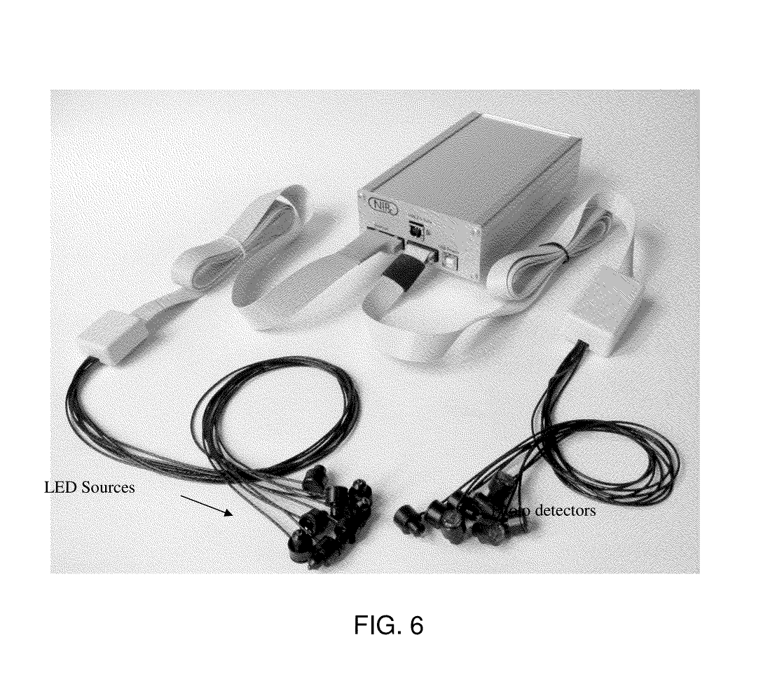

FIG. 5 provides a bird's eye view of another exemplary tumor detector according to an embodiment of the present disclosure. The brassier 1000 includes a right-side cup 100R, a left-side cup 100L, a torso strap 200, an electronics unit 300, a pair of shoulder straps 210, a pair of shoulder strap hooking elements 220, and at least one torso strap hooking element 320. A light emitting device (not expressly shown; See FIG. 6) incorporating a plurality of emission points and a light sensing device (not expressly shown; See FIG. 6) incorporating a plurality of detection points are embedded within the brassier 1000. Emission/detection points 100 are schematically illustrated as circles located on the cups (100R, 100L) of the brassier 1000.

The right-side cup 100R and the left-side cup 100L can be rigid structures on which the emission/detection points 100 are arranged. The relative distance between each pair of emission/detection points 100 on a cup (100R or 100L) remain invariant during usage of the brassier 1000, and especially during a tomographic scanning process. Each cup (100R or 100L) functions as a stationary platform on which the light emitting device (including the plurality of emission points) and the light sensing device (including the plurality of detection points). The emission points can be physically implemented as light emitting diodes (LED's). The detection points can be physically implemented as light-sensing detectors as known in the art.

The torso strap 200 connects the right-side cup 100R and the left-side cup 100L, and is made of a pliable material that can laterally wrap around the chest portion of the user of the exemplary tumor detector. A portion of the torso strap 200 can be attached to the electronic unit 300. At least one torso strap hooking element 320 can be provided at one end of the torso strap 200 to enable interlocking with at least one torso strap interlocking mechanism 321 located on the electronics unit.

The pair of shoulder straps 210 can be a pair of flexible strings of suitable length for providing additional structural support to the tumor detector 1000 while mounted on the user of the tumor detector 100. The pair of shoulder strap hooking elements 220 can be provided at the ends of the pair of shoulder straps 210 to be attached to a pair of shoulder strap interlocking mechanisms 321 located on the electronics unit 300. The length of the pair of shoulder straps 210 can be adjustable.

The electronics unit 300 can include a controller configured to control timing of light emission at the plurality of emission points and to collect data measured by the light sensing device. The electronics unit 300 can optionally include a computing means configured to generate at least one tomographic image, wherein the at least one tomographic image includes at least one of a two-dimensional map and a three-dimensional map of human breasts. As used herein, a computing means refers to any device capable of performing mathematical calculations on the data collected by the controller so that at least one image of the scanned breasts can be generated. The computing means can be any portable computing device such as a laptop computer or a dedicated computing unit capable of performing graphic operations on data. The electronics unit 300 can be optionally equipped with a communications unit capable of transmitting the data, either in graphical formats or in non-graphical formats, to a display device. The display device may be attached to the electronics unit 300, or can be located at a remote location such as a doctor's office. Further, the electronics unit 300 can include a power supply system for providing power to the electronics circuitry therein.

In general, a tumor detector 100 of the present disclosure includes a brassiere 1000 conforming to a pair of human breasts, a light emitting device (See FIG. 6) configured to emit light at a plurality of emission points (a first subset of emission/detection points 100) on the brassiere 1000, a light sensing device configured to detect light at a plurality of detection points (a second subset of the emission/detection points 100) on the brassiere 1000, and a controller (located within the electronics unit 300) configured to control timing of light emission at the plurality of emission points and to collect data measured by the light sensing device. The light emitting device and the light sensing device can be embedded within the cups (100R, 100L) and the torso strap 200. In case fiber optics cables are employed to transmit light, a portion of the light emitting device that generates the light may be located within the electronics unit 300 provided that the light can be transmitted to the plurality of emission points on the cups (100R, 100L) through the fiber optics cables.

In one embodiment, the tumor detector 1000 can further include a computing means (embedded within the electronics unit 300) attached to the brassiere 1000. The computing means can be physically attached to, and can be a part of, the brassiere 1000. The computing means can be provided within the electronics unit 300. Additionally or alternately, the computing means can be configured to generate at least one tomographic image. The at least one tomographic image can include at least one of a two-dimensional map and a three-dimensional map of human breasts. The tumor detector of the present disclosure can further include a display device (See FIG. 4) configured to display the at least one tomographic image through data transmission from the computing device to the display device. Additionally or alternately, the computing means can be configured to transmit the at least one tomographic image to a medical facility via wireless communication or via wired communication.

In another embodiment, the computing means can be configured to generate at least one image for optical density in a breast among the pair of human breasts (i.e., in one of the user's own breasts) employing data from another breast among the pair of human breasts as a base line for determining presence or absence of anomalous deviations in optical density in the breast. In this case, the computing means can be configured to compare a left-breast image from a left breast within the pair of human breasts with a right-breast image from a right breast within the pair of human breasts. Because most females have substantial symmetry between a left breast and a right breast in terms of the density and structure of the materials of the breasts, the self-referencing method can be a reliable method for establishing references for detecting tumors for most individuals. Further, if a substantial asymmetry between the sizes of the left breast and the right breast is known, a user-defined parameter can be input into the electronics unit 300 to mitigate the effect of the size differences between the left breast and the right breast. Alternately or additionally, size differences within a normal range between the left breast and the right breast can be accommodated by an algorithm within an automated program for processing the measured data so as to minimize skewing of the data due to the size differences between an individual's breasts.

The computing means can be further configured to generate a map of regions in which local variations in optical density of materials in each of the pair of human breasts are illustrated. For example, the left-breast image can be generated employing data from the right breast as reference data, and the right-breast image can be generated employing data from the left breast as reference data. The particular configurations of the computing means can be accomplished by providing a specific automatic program to collect the data, and to compensate for the differences in the breast size, and to generate at least one map representing the variations in the composition of the breasts based on the measured variations in the optical density of the material within the breasts. In one embodiment, the computing means can be configured to generate a pair of images corresponding to each of the pair of breasts, and to determine if one of the pair of images differ from another of the pair of images by more than a predefined threshold for identifying presence of a tumor.

In one embodiment, the light emitting device can be configured to emit near infrared radiation having a wavelength in a range from 800 nm to 2,500 nm. The light sensing device can be optimized for detection of the radiation at the wavelength of the emitted near infrared radiation.

In yet another embodiment, the tumor detector can further include a portable power supply device attached to, and optionally included within, the brassier 1000 and providing power to the light emitting device and the controller. In one embodiment, the portable power supply device may be located within the electronics unit 300, may be attached to, or located within, the torso strap 200 of the brassiere 1000. Further, the portable power supply device may include a rechargeable battery or a non-rechargeable battery.

In still yet another embodiment, the plurality of emission points and the plurality of detection points can be fixed locations on the brassiere. Thus, the relative distance between each pair of emission/detection points 100 on a same cup (100R or 100L) can be the same during the tomographic scanning process. The plurality of emission points and the plurality of detection points may have a mirror symmetry between the right-side cup 100R and the left-side cup 100L.

In one embodiment, the emission/detection points 100 on each cup (100R or 100L) may be divided into different groups located within different quadrants. As used herein, a quadrant of a cup (100R or 100L) refers to one of the four regions defined by a horizontal plane HP passing through the most protruding point of the cup (100R or 100L) and by a vertical plane passing through the most protruding point of the cup (100R or 100L). The intersection of the horizontal plane HP and the vertical planes with the cups (100R, 100L) are illustrated as dotted lines in FIG. 5. The most protruding point of each cup (100R or 100L) is herein referred to as an “apex” of the cup.

As used herein, a first quadrant Q1 refers to the portion of each cup (100R or 100L) that is located above the horizontal plane HP and is more proximal to the center of mass CM of the cups (100R, 100L) than the vertical plane passing through the apex (101R or 101L) of the cup (100R, 100L). A second quadrant Q2 refers to the portion of each cup (100R or 100L) that is located above the horizontal plane HP and more distal from the center of mass CM of the cups (100R, 100L) than the vertical plane passing through the apex (101R or 101L) of the cup (100R, 100L). A third quadrant Q3 refers to the portion of each cup (100R or 100L) that is located below the horizontal plane HP and is more distal from the center of mass CM of the cups (100R, 100L) than the vertical plane passing through the apex (101R or 101L) of the cup (100R, 100L). A fourth quadrant Q4 refers to the portion of each cup (100R or 100L) that is located below the horizontal plane HP and more proximal to the center of mass CM of the cups (100R, 100L) than the vertical plane passing through the apex (101R or 101L) of the cup (100R, 100L). As used herein, a “center of mass” of an object refers to the canter of mass as defined in physics.

In one embodiment, the plurality of emission points (which is a first subset of the emission/detection points 100) and the plurality of detection points (which is a second subset of the emission/detection points 100) can be located on opposing sides of each cup (100R, 100L) within the brassiere 1000. Such configurations enhance signal to noise ratio by requiring that all direct optical signal paths between emission points and detections points intersect a portion of a breast.

In one example, the plurality of emission points is located above a horizontal plane HP passing through apexes (101R or 101L) of the cups (100R, 100L), and the plurality of detection points is located below the horizontal plane HP. In this case, the plurality of emission points can be located within the first and second quadrants (Q1, Q2), and the plurality of detection points can be located within the third and fourth quadrants (Q3, Q4).

In another example, the plurality of emission points is located below a horizontal plane HP passing through apexes (101R or 101L) of the cups (100R, 100L), and the plurality of detection points is located above the horizontal plane HP. In this case, the plurality of emission points can be located within the third and fourth quadrants (Q3, Q4), and the plurality of detection points can be located within the first and second quadrants (Q1, Q2).

In yet another example, the plurality of emission points is more proximal to the center of mass CM of the cups (100R, 100L) than the apexes (101R or 101L) of the cups (100R, 100L) are to the center of mass CM, and the plurality of detection points is more distal from the center of mass CM than the apexes (101R or 101L) of the cups are from the center of mass CM. In this case, the plurality of emission points can be located within the first and fourth quadrants (Q1, Q4), and the plurality of detection points can be located within the second and third quadrants (Q2, Q3).

In still another example, the plurality of emission points is more distal from the center of mass CM of the cups (100R, 100L) than the apexes (101R or 101L) of the cups (100R, 100L) are from the center of mass CM, and the plurality of detection points is more proximal to the center of mass CM than the apexes (101R or 101L) of the cups (100R, 100L) are to the center of mass CM. In this case, the plurality of emission points can be located within the second and third quadrants (Q2, Q3), and the plurality of detection points can be located within the first and fourth quadrants (Q1, Q4).

The tumor detector of the present disclosure can be manufactured with different cup sizes. Upon selection of the correct sup size that fits the breasts of the user (or the test subject if another person operates the device for the test subject), the tumor detector of the present disclosure can be mounted on the breasts of the user, and can be operated to perform a tomographic scan process. Thus, the tumor detector of the present disclosure enables self-diagnosis of breast cancer in the privacy of the user's choosing. For example, the tumor detector of the present disclosure can be operated in any non-medial facility such as the user's home, or at public places such as parks and beaches. In one embodiment, the tumor detector can be a portable device configured to be operated without external power supply while mounted on the user's breasts.

In one embodiment, the tumor detector of the present disclosure can include a computing means attached to the brassiere 100, and the tomographic scan process can be controlled employing an automatic program stored in the computing means. Upon completion of a tomographic scanning, at least one tomographic image can be generated based on the result of the tomographic scan process as collected by the controller and processed by the computing means. The at least one tomographic image includes at least one of a two-dimensional map and a three-dimensional map of human breasts. The at least one tomographic image can be displayed on a display device through data transmission from the computing device to the display device.

The tumor detector of the present disclosure enables detection of presence, or absence, of a tumor in human breasts. The assembly of the brassier 1000, a light emitting device located on the brassiere 1000, and a light sensing device located on the brassiere 1000 is provided. The light emitting device is configured to emit light at a plurality of emission points on the brassiere 1000, and the light sensing device is configured to detect light at a plurality of detection points on the brassiere 1000. The assembly is disposed on, i.e., mounted on, a pair of human breasts. The pair of human breasts is illuminated with infrared radiation from the light emitting device. Radiation is detected at the plurality of detection points employing the light sensing device. A pair of images from the pair of human breasts can be compared, and employed as a reference for the other image, to determine if one of the pair of images differ from another of the pair of images by more than a predefined threshold for identifying presence of a tumor. The predefined threshold for identifying presence of a tumor can be preprogrammed into a tumor detection program running on the computing means. The predefined threshold can be determined by comparing images of test subjects in a laboratory setting, and can be provided to the automated program as an input. The timing of light emission at the plurality of emission points can be controlled employing the controller. Data measured by the light sensing device can be collected employing the controller.

In addition, whereas light delivery and collection could be achieved using intervening optical fibers, it is practical to introduce LED's in contact with the breast, as well as silicon photodiode detectors with limited amplification circuitry also in contact with the breast. Shown in FIG. 6 is a photograph of an exemplary compact NIRS system that supports a multi-element, fibre-free sensing arrangement. Lead wires from the active optical source and detectors are confined within the brassiere and terminate to a connector that leads to a compact amplifier. The amplifier contains timing circuitry and additional amplification capabilities similar to the type described by Schmitz et al. (2002), supra. In the case where more than one optical source is employed, illumination is achieved using a time-multiplexing scheme. Also positioned within the brassiere is an accelerometer to detect excessive subject motion.

Having implemented the considered sensing arrangement and positioned the brassiere in contact with the breasts, a time series measurement is performed lasting on the order of 5 minutes, sufficient to determine the amplitude of the low frequency elements of the vasomotor signal. Having performed the optical measurement, collected data can be evaluated using any of a number of strategies that serve to evaluate the temporal variability of the measured signal. In the case where at least two illuminating wavelengths are employed, it is desirable to compute components of the hemoglobin signal using a modified Beer-Lambert law as described by Schmitz et al. (2005), supra. The derived biomarker can comprise measures of temporal variance of the time series measure from either a single component of the hemoglobin signal (e.g. oxyhemoglobin) or combinations of elements (e.g., deoxyhemoglobin, oxyhemoglobin, total hemoglobin, hemoglobin oxygen saturation, oxygen extraction efficiency) or other metrics that involve additional data transformations intended to form a 2D or 3D image of the breast. Having obtained a suitable biomarker, the measure can be used directly in reference to a healthy or breast cancer free population or could be compared to the contralateral breast to yield a secondary metric whose value is compared to a control group. Regardless of the details of the derived biomarker, the amplitude of the signal will serve to determine a diagnostic threshold above which significant evidence of the presence of cancer is considered present.

It is expressly understood that the biological origin of the temporal variance of the measured response is unlikely to be confined to the limits of the tumor boarder and thus will not usefully serve to determine tumor size. Nevertheless, the measured signal will serve as a reliable indicator for the presence of a cancerous tumor. The considered method recognizes the inherent advantage of NIR measures of vascular pulsatility compared to previously considered IR measures and recognizes that the biology of NO production in tumors, combined with known changes in the functional anatomy involving the tumor vasculature favor detection of a spatially dispersed signal highly specific to the presence of breast cancer.

While the invention has been described in terms of specific embodiments, it is evident in view of the foregoing description that numerous alternatives, modifications and variations will be apparent to those skilled in the art. Each of the various embodiments of the present disclosure can be implemented alone, or in combination with any other embodiments of the present disclosure unless expressly disclosed otherwise or otherwise impossible as would be known to one of ordinary skill in the art. Accordingly, the invention is intended to encompass all such alternatives, modifications and variations which fall within the scope and spirit of the invention and the following claims.

What is claimed is:

1. A tumor detector comprising:
a brassiere conforming to a pair of human breasts;
a light emitting device configured to emit light at a plurality of emission points on said brassiere;
a light sensing device configured to detect light at a plurality of detection points on said brassiere; and
a controller configured to control timing of light emission at said plurality of emission points and to collect data measured by said light sensing device.

2. The tumor detector of claim 1, further comprising a computing means attached to said brassiere and configured to is configured to generate at least one tomographic image.

3. The tumor detector of claim 2, wherein said computing means is physically attached to said brassiere.

4. The tumor detector of claim 2, wherein said at least one tomographic image comprises at least one of a two-dimensional map and a three-dimensional map of human breasts.

5. The tumor detector of claim 4, further comprising a display device configured to display said at least one tomographic image through data transmission from said computing device to said display device.

6. The tumor detector of claim 4, wherein said computing means is configured to transmit said at least one tomographic image to a medical facility via wireless communication or via wired communication.

7. The tumor detector of claim 2, the computing means is configured to generate at least one image for optical density in a breast among said pair of human breasts employing data from another breast among said pair of human breasts as a base line for determining presence or absence of anomalous deviations in optical density in said breast.

8. The tumor detector of claim 2, wherein said computing means is configured:
to compare a left-breast image from a left breast within said pair of human breasts with a right-breast image from a right breast within said pair of human breasts, and
to generate a map of regions in which local variations in optical density of materials in each of said pair of human breasts are illustrated.

9. The tumor detector of claim 8, wherein said left-breast image is generated employing data from said right breast as reference data, and said right-breast image is generated employing data from said left breast as reference data.

10. The tumor detector of claim 2, wherein said computing means is configured to:
generate a pair of images corresponding to each of said pair of breasts; and
determine if one of said pair of images differ from another of said pair of images by more than a predefined threshold for identifying presence of a tumor.

11. The tumor detector of claim 1, wherein said light emitting device is configured to emit near infrared radiation having a wavelength in a range from 800 nm to 2,500 nm.

12. The tumor detector of claim 1, further comprising a portable power supply device attached to said brassier and providing power to said light emitting device and said controller.

13. The tumor detector of claim 1, wherein said plurality of emission points and said plurality of detection points are fixed location on said brassiere.

14. The tumor detector of claim 1, wherein said plurality of emission points and said plurality of detection points are located on opposing sides of each cup within said brassiere.

15. The tumor detector of claim 14, wherein said plurality of emission points is located above a horizontal plane passing through apexes of said cups, and said plurality of detection points is located below said horizontal plane.

16. The tumor detector of claim 14, wherein said plurality of emission points is located below a horizontal plane passing through apexes of said cups, and said plurality of detection points is located above said horizontal plane.

17. The tumor detector of claim 1, wherein said tumor detector is a portable device configured to be operated without external power supply while mounted on a person.

18. A method of performing a tomographic scan process, said method comprising:
providing a tumor detector of claim 1; and
performing a tomographic scan process employing said tumor detector.

19. A method of detecting presence of a tumor in human breasts, said method comprising:
providing an assembly of a brassiere, a light emitting device located on said brassiere, and a light sensing device located on said brassiere, wherein said light emitting device is configured to emit light at a plurality of emission points on said brassiere, and said light sensing device is configured to detect light at a plurality of detection points on said brassiere;
disposing said assembly on a pair of human breasts;
illuminating said pair of human breasts with near infrared radiation from said light emitting device;
detecting radiation at said plurality of detection points employing said light sensing device; and
comparing a pair of images from said pair of human breasts to determine if one of said pair of images differ from another of said pair of images by more than a predefined threshold for identifying presence of a tumor.

20. The method of claim 19, further comprising:
controlling timing of light emission at said plurality of emission points employing a controller; and
collecting data measured by said light sensing device employing said controller.
